(19) United States
(12) Patent Application Publication
(10) Pub. No.: US 2013/0035232 A1
(43) Pub. Date: Feb. 7, 2013

(54) INDUCTION AND STABILIZATION OF ENZYMATIC ACTIVITY IN MICROORGANISMS

(71) Applicants: George E. Pierce, Canton, GA (US); Trudy Ann Tucker, Atlanta, GA (US)

(72) Inventors: George E. Pierce, Canton, GA (US); Trudy Ann Tucker, Atlanta, GA (US)

(73) Assignee: GEORGIA STATE UNIVERSITY RESEARCH FOUNDATION, INC., Atlanta, GA (US)

(21) Appl. No.: 13/574,943

(22) PCT Filed: Jan. 24, 2011
(86) PCT No.: PCT/US11/22278

§ 371 Date: Oct. 23, 2012

Related U.S. Application Data
(60) Provisional application No. 61/297,897, filed on Jan. 25, 2010.

(51) Int. Cl. C12N 9/88; A01N 63/00; C12N 9/78; C12N 9/82; C12N 1/20; C12N 11/00

(52) U.S. Cl. 504/117; 435/253.2; 435/253.3; 435/252.1; 435/174; 435/232; 435/227; 435/229

(57) ABSTRACT

Provided herein are methods for inducing and stabilizing an enzyme activity. Optionally, the enzyme is in a microorganism capable of producing the enzyme. In some embodiments, the enzyme can be nitrile hydratase, amidase, or asparaginase I. Provided are compositions comprising enzymes or microorganisms having induced and/or stabilized activity. Also provided are methods of delaying a plant development process by exposing a plant or plant part to the enzymes or microorganisms having induced and/or stabilized activity.

### BACKGROUND

Microorganisms, and their enzymes, have been utilized as biocatalysts in the preparation of various products. The action of yeast in the fermentation of sugar to ethanol is an immediately recognizable example. In recent years, there has been a growing interest in the use of microorganisms and their enzymes in commercial activities not normally recognized as being amenable to enzyme use. One example is the use of microorganisms in industrial processes, particularly in the treatment of waste products.

Stability, which is a key element for a practical biological catalyst, has been a significant hurdle to using nitrile hydratase and/or amidase in many commercial applications. While immobilization and chemical stabilizing agents are recognized approaches for improving enzyme stability, the current immobilization and stabilization techniques are still in need of further development.

### SUMMARY

Provided herein are methods for inducing and stabilizing an enzyme activity. Optionally, the enzyme is in a microorganism capable of producing the enzyme. In some embodiments, the enzyme can be nitrile hydratase, amidase, or asparaginase I. Provided are compositions comprising enzymes or microorganisms having induced and/or stabilized activity. Also provided are methods of delaying a plant development process by exposing a plant or plant part to the enzymes or microorganisms having induced and/or stabilized activity.

The details of one or more aspects are set forth in the accompanying drawings and description below. Other features, objects, and advantages will be apparent from the description and drawings and from the claims.

### DESCRIPTION OF DRAWINGS

FIG. 1 shows a graph demonstrating the stabilizing effect on nitrile hydratase activity provided by immobilization in calcium alginate.

FIG. 2 shows a graph demonstrating the stabilizing effect on nitrile hydratase activity provided by immobilization in polyacrylamide.

FIG. 3 shows a graph demonstrating the stabilizing effect on nitrile hydratase activity provided by immobilization in hardened, polyethyleneimine cross-linked calcium alginate or polyacrylamide.

FIG. 4 shows a graph demonstrating the stabilizing effect on nitrile hydratase activity provided by immobilization through glutaraldehyde cross-linking.

FIG. 5 shows a graph demonstrating the asparaginase I activity in Rhodococcus sp. DAP 96253 cells induced with asparagine.

FIG. 6 shows a graph demonstrating the stabilizing effect on nitrile hydratase activity at 55° C. in Rhodococcus sp. DAP 96253 cells grown on YEMEA supplemented with glucose, fructose, maltose, maltodextrin and induced with cobalt and urea.

FIG. 7 shows a graph demonstrating the stabilizing effect on nitrile hydratase activity at 55° C. in Rhodococcus sp. DAP 96253 cells grown on YEMEA supplemented with glucose, fructose, maltose, maltodextrin; induced with cobalt and urea; and stabilized with trehalose.

### DETAILED DESCRIPTION

As used herein, the singular forms “a”, “an”, “the”, include plural referents unless the context clearly dictates otherwise.

Throughout the specification the word “comprising,” or grammatical variations thereof, will be understood to imply the inclusion of a stated element, integer or step, or group of elements, integers or steps, but not the exclusion of any other element, integer or step, or group of elements, integers or steps.

Provided herein are methods for inducing and stabilizing enzymatic activity in microorganisms through the use of media and compositions comprising trehalose and, optionally, amide containing amino acids. Generally, nitrile hydratase producing microorganisms are used for inducing the production of a number of useful enzymes. For example, provided herein is a method for inducing an enzyme activity selected from the group consisting of nitrile hydratase activity, amidase activity, asparaginase I activity and combinations thereof in a nitrile hydratase producing microorganism comprising culturing the nitrile hydratase producing microorganism in a medium comprising trehalose and, optionally, one or more amide containing amino acids.

Further provided are methods for improving the stabilization of various enzymes, such as nitrile hydratase, asparaginase I, and amidase. For example, provided is a method for stabilizing desired enzyme activity in an enzyme or a microorganism capable of producing the enzyme comprising contacting the enzyme or microorganism capable of producing the enzyme with a composition comprising trehalose and one or more amide containing amino acids, wherein the enzyme is selected from the group consisting of nitrile hydratase, amidase and asparaginase I.

Provided are bio-detoxifying catalysts (particularly incorporating enzymes, such as nitrile hydratase and amidase) that can maintain a commercially useful level of enzymatic activity over time. The bio-detoxifying catalysts are particularly characterized in that the enzymatic activity of the biocatalysts can be induced and stabilized by their environment, as described herein.

The methods disclosed herein can be used to induce enzymatic activity that is both of a level and stability that is useful in a practical bio-detoxifying catalyst. The methods are further characterized by the ability to induce higher levels of asparaginase I from microorganisms, including (but not limited to) Gram-positive microorganisms, and to improve the stability of such asparaginase I activity.

Enzymatic activity, as used herein, generally refers to the ability of an enzyme to act as a catalyst in a process, such as the conversion of one compound to another compound. Likewise, the desired activity referred to herein can include the activity of one or more enzymes being actively expressed by one or more microorganisms. In particular, nitrile hydratase catalyzes the hydrolysis of nitrile (or cyanohydrin) to the corresponding amide (or hydroxy acid). Amidase catalyzes the hydrolysis of an amide to the corresponding acid or hydroxy acid. Similarly, an asparaginase enzyme, such as asparaginase I, catalyzes the hydrolysis of asparagine to aspartic acid.

Activity can be referred to in terms of “units” per mass of enzyme or cells (typically based on the dry weight of the cells, e.g., units/mg cdw). A “unit” generally refers to the ability to convert a specific content of a compound to a different compound under a defined set of conditions as a function of time. Optionally, one “unit” of nitrile hydratase activity can relate to the ability to convert 1 μmol of acrylonitrile to its corresponding amide per minute, per milligram of cells (dry weight) at a pH of 7.0 and a temperature of 30° C. Similarly, one unit of amidase activity can relate to the ability to convert 1 μmol of acrylamide to its corresponding acid per minute, per milligram of cells (dry weight) at a pH of 7.0 and a temperature of 30° C. Further, one unit of asparaginase I activity can relate to the ability to convert 1 μmol of asparagine to its corresponding acid per minute, per milligram of cells (dry weight) at a pH of 7.0 and a temperature of 30° C.

The methods are particularly advantageous in that induction and stabilization of the microorganism can be accomplished without the requirement of introducing hazardous nitriles, such as acrylonitrile, into the environment. Previously, it was believed that induction of specific enzyme activity in certain microorganisms required the addition of chemical inducers. For example, in the induction of nitrile hydratase activity in Rhodococcus rhodochrous and Pseudomonas chloroaphis, it was generally necessary to supplement with hazardous chemicals, such as acetonitrile, acrylonitrile, acrylamide, and the like. It has been discovered that high enzymatic activity in nitrile hydratase producing microorganisms can be induced and stabilized with the use of non-hazardous media additives, such as trehalose and, optionally, amide containing amino acids, and derivatives thereof. Optionally, asparagine, glutamine, or combinations thereof, can be used as inducers with the complete exclusion of hazardous chemicals, such as acetonitrile, acrylonitrile, acrylamide, and the like. Thus, provided are safer methods for production of commercially useful enzymes and microorganisms and their use in further methods, such as for detoxifying waste streams. Safer methods of inducing and stabilizing enzymatic activity in microorganisms are described in U.S. Pat. No. 7,531,343 and U.S. Pat. No. 7,531,344, which are incorporated herein by reference.

Preferably, the disclosed methods provide for significant increases in the production and stability of a number of enzymes, and the microorganisms capable of producing the enzymes, using modified media, immobilization, and stabilization techniques, as described herein. For example, induction and stabilization can be increased through use of media comprising trehalose and, optionally, amide-containing amino acids, or derivatives thereof.

Nitrile hydratase producing microorganisms for use in the methods provided herein include, but are not limited to, bacteria selected from the group consisting of genus Pseudomonas, genus Rhodococcus, genus Brevibacterium, genus Pseudonocardia, genus Nocardia, and combinations thereof. Optionally, the nitrile hydratase producing microorganism is from the genus Rhodococcus. Optionally, the microorganism from the genus Rhodococcus is Rhodococcus rhodochrous DAP 96622, Rhodococcus sp. DAP 96523 or combinations thereof. Exemplary organisms include, but are not limited to, Pseudomonas chloroaphis (ATCC 43051) (Gram positive), Pseudomonas chloroaphis (ATCC 13985) (Gram positive), Rhodococcus erythropolis (ATCC 47072) (Gram positive), and Brevibacterium ketoglutamicum (ATCC 21533) (Gram positive). Examples of Nocardia and Pseudonocardia species have been described in European Patent No. 0790310; Collins and Knowles J. Gen. Microbiol. 129:711-718 (1983); Harper Biochem. J. 165:309-319 (1977); Harper Int. J. Biochem. 17:677-683 (1985); Linton and Knowles J. Gen. Microbiol. 132:1493-1501 (1986); and Yamaki et al., J. Ferm. and Bioeng. 83:474-477 (1997).

Methods for cultivating microorganisms, particularly nitrile hydratase producing microorganisms, for inducing a desired enzyme activity in the microorganisms are provided. In some embodiments, the methods comprise culturing a nitrile hydratase producing microorganism in a medium comprising trehalose and, optionally, one or more amide containing amino acids or derivatives thereof. Optionally, disclosed is a method for inducing nitrile-detoxification activity using a medium supplemented with trehalose and, optionally, amide containing amino acids or derivatives thereof, which preferably include asparagine, glutamine or a combination thereof. More particularly, the methods comprise culturing the microorganism in the medium and optionally collecting the cultured microorganisms or enzymes produce by the microorganisms.

The disclosed methods are particularly useful for inducing a desired enzyme activity. Many types of microorganisms, including those described herein, are capable of producing a variety of enzymes having a variety of activities. As is generally understood in the art, the type of enzyme activity induced in microorganism cultivation can vary depending upon the strain of microorganism used, the method of growth used, and the supplementation used with the growth media. The methods and compositions disclosed herein allow for the induction of a variety of enzyme activities through the use of trehalose and, optionally, amide containing amino acids, or derivatives thereof. Optionally, the disclosed methods and compositions allow for the induction of one or more enzymes selected from the group consisting of nitrile hydratase, amidase, and asparaginase I.

In some embodiments, the disclosed methods and compositions allow for the simultaneous induction of both nitrile hydratase and amidase. This is useful, for example, for industrial applications, such as the treatment of nitrile-containing waste streams. Such treatment requires a first treatment to convert nitriles to amides and a second treatment to convert amides to acids. The ability to simultaneously produce nitrile hydratase and amidase removes the need to separately prepare the enzymes and allows for a single treatment step.

In the provided methods, induction and stabilization of enzymes can be achieved without the use of hazardous nitriles. The induction of many types of enzyme activity, such as nitrile hydratase activity, has traditionally included supplementation with nitriles, such as acetonitrile, acrylonitrile, succinonitrile, and the like. Moreover, if multiple induction was desired (i.e., induction of activity in a single enzyme to degrade two or more types of nitriles), it was generally necessary to include two or more types of hazardous nitriles. The disclosed methods, arising from the use of trehalose and/or one or more amide containing amino acids or derivatives thereof as enzymatic inducers and stabilizers, eliminates the need for hazardous chemicals to facilitate single or multiple enzymatic induction. Particularly, the methods herein are beneficial in that multiple induction and stabilization is possible through the use of trehalose and/or one or more amide containing amino acids or derivatives thereof in the culture medium or mixture. Thus, the disclosed methods are particularly useful for preparing an enzyme or microorganism having activity for degrading a plurality of nitrile containing compounds. Moreover, the methods provide the ability to detoxify a variety of nitriles or amides, such as nitriles having a single C≡N moiety, dinitriles (compounds having two C≡N moieties), or compounds having multiple nitrile moieties (e.g., acrolein cyanohydrin). Such enzymes, or microorganisms, are herein referred to as being multiply induced.

While the disclosed methods eliminate the need for hazardous chemicals for enzyme activity induction, the use of such further inducers is not excluded. For example, one or more nitriles could be used to assist in specific activity development. Media supplemented with succinonitrile and cobalt can be useful for induction of enzymes, including, for example, nitrile hydratase, amidase and asparaginase I. However, the use of nitriles is not necessary for induction of enzyme activity. While the use of nitriles and other hazardous chemicals is certainly not preferred, optionally, such use is possible.

Optionally, the methods and compositions are particularly characterized by the ability to induce a desired activity that is greater than possible using previously known methods. Using the methods provided herein, the induced nitrile hydratase producing microorganism has an enzyme activity greater than or equal to the activity of the same enzyme when induced in a medium comprising a nitrile containing compound. By way of example, the induced nitrile hydratase producing microorganism has an enzyme activity that is at least 5% greater than the activity of the same enzyme when induced in a medium comprising a nitrile containing compound. Optionally, the nitrile hydratase activity produced is at least 10%, at least 12%, or at least 15% greater than the activity produced in the same microorganism by induction with a nitrile containing compound.

Commercial use of enzymes for the treatment of waste water, as well as other commercial uses of various enzymes, is generally limited by the instability of the induced activity. For examples, fresh cells will typically lose at least 50% of their initial activity within 24 hours at a temperature of 25° C. Thus, when cells are to be used as a catalyst, the cells must be made at the time of need and cannot be stored for future use. Nitrile hydratase activity can be stabilized through addition of nitrile containing compounds; however, this again necessitates the use of undesirable, hazardous chemicals. The disclosed methods and compositions solve this problem. For example, cells having induced nitrile hydratase activity can be stabilized without the need for hazardous chemicals, such that the cells have a viable enzyme activity for a time period of up to one year. Thus, the disclosed methods and compositions stabilize enzymes, or microorganisms capable of producing such enzymes, such that the activity of the enzyme is extended well beyond the typical period of useful activity.

Thus, provided are methods for stabilizing a desired activity in an enzyme or a microorganism capable of producing the enzyme. Such methods comprise contacting the enzyme, or a microorganism capable of producing the enzyme, with trehalose and, optionally, one or more amide containing amino acids. The trehalose and amide containing amino acids or derivatives thereof can, for example, be added to the microorganisms at the time of culturing the microorganisms or can be added to a mixture comprising the microorganisms or enzymes. Optionally, the desired activity of the enzyme or microorganism capable of producing the enzyme is stabilized such that the desired activity after a time of at least 30 days at a temperature of 25° C. is maintained at a level of at least about 50% of the initial activity exhibited by the enzyme or the microorganism capable of producing the enzyme.

Further stabilization can be achieved through immobilization methods, such as affixation, entrapment, and cross-linking, thereby, extending the time during which enzyme activity can be used. Thus, the methods further comprise at least partially immobilizing the microorganism. Stabilization can be provided by immobilizing the enzymes or microorganisms producing the enzymes. For example, enzymes harvested from the microorganisms or the induced microorganisms themselves can be immobilized to a substrate as a means to stabilize the induced activity. Optionally, the nitrile hydratase producing microorganisms are at least partially immobilized. Optionally, the enzymes or microorganisms are at least partially entrapped in or located on the surface of a substrate. This allows for presentation of an immobilized material with induced activity (e.g., a catalyst) in such a manner as to facilitate reaction of the catalyst with an intended material and recovery of a desired product while simultaneously retaining the catalyst in the reaction medium and in a reactive mode.

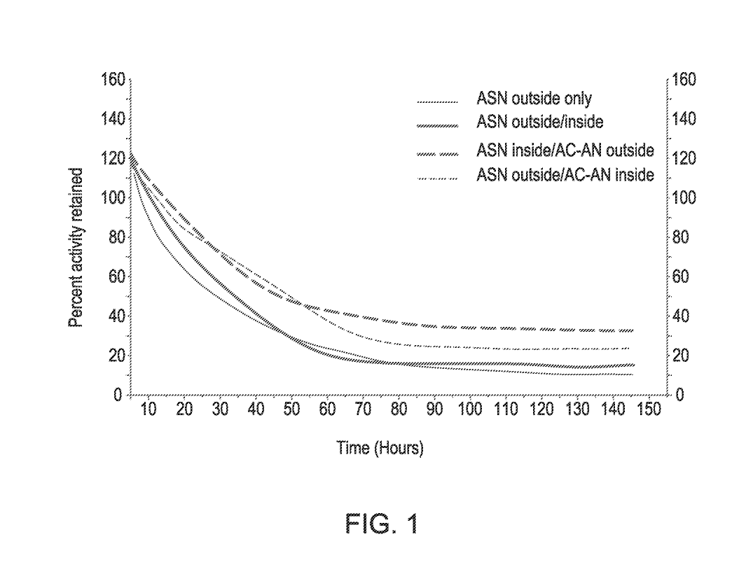

Any substrate generally useful for affixation of enzymes or microorganisms can be used. Optionally, the substrate comprises alginate or salts thereof. Alginate is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), alternating M and G-residues (MG-blocks), or randomly organized blocks. Optionally, calcium alginate is used as the substrate. The calcium alginate can, for example, be cross-linked, such as with polyethyleneimine, to form a hardened calcium alginate substrate. Further description of such immobilization techniques can be found in Bucke, “Cell Immobilization in Calcium Alginate,” Methods in Enzymology, vol. 135, Part B (ed. K. Mosbach) pp. 175-189 (1987), which is incorporated herein by reference. The stabilization effect of immobilization using polyethyleneimine cross-linked calcium alginate is illustrated in FIG. 1 and is further described in Example 2.

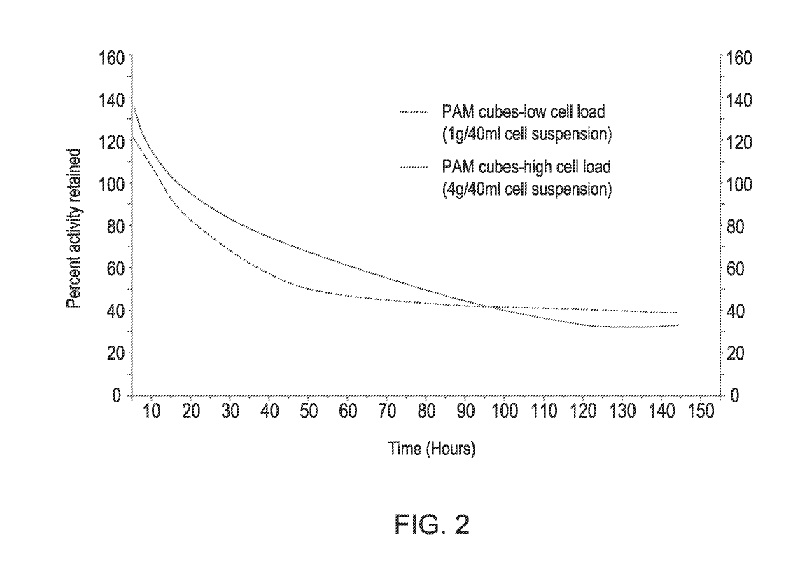

Optionally, the substrate comprises an amide-containing polymer. Any polymer comprising one or more amide groups can be used. Optionally, the substrate comprises a polyacrylamide polymer. The stabilization effect of immobilization using polyacrylamide is illustrated in FIG. 2, which is further described in Example 3.

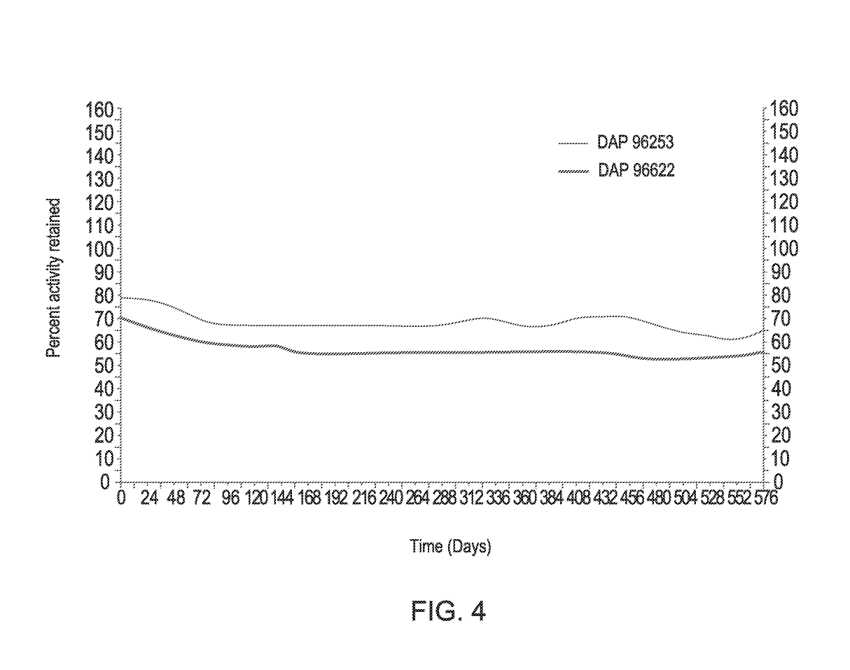

Stabilization can further be achieved through cross-linking. For example induced microorganisms can be chemically cross-linked to form agglutinations of cells. Optionally, the induced microorganisms are cross-linked using glutaraldehyde. For example, microorganisms can be suspended in a mixture of de-ionized water and glutaraldehyde followed by addition of polyethyleneimine until maximum flocculation is achieved. The cross-linked microorganisms (typically in the form of particles formed of a number of cells) can be harvested by simple filtration. Further description of such techniques is provided in Lopez-Gallego, et al., J. Biotechnol. 119:70-75 (2005), which is incorporated herein by reference. The stabilization effect of glutaraldehyde cross-linking is illustrated in FIG. 4 and is further described in Example 5.

Optionally, the microorganisms can be encapsulated rather than allowed to remain in the classic Brownian motion. Such encapsulation facilitates collection, retention, and reuse of the microorganisms and generally comprises affixation of the microorganisms to a substrate. Such affixation can also facilitate stabilization of the microorganisms, as described above, or may be solely to facilitate ease of handling of the induced microorganisms or enzymes.

The microorganisms can be immobilized by any method generally recognized for immobilization of microorganisms, such as sorption, electrostatic bonding, covalent bonding, and the like. Generally, the microorganisms are immobilized on a solid support which aids in the recovery of the microorganisms from a mixture or solution, such as a detoxification reaction mixture. Suitable solid supports include, but are not limited to granular activated carbon, compost, wood or wood products, (e.g., paper, wood chips, wood nuggets, shredded pallets or trees), metal or metal oxide particles (e.g., alumina, ruthenium, iron oxide), ion exchange resins, DEAE cellulose, DEAE-SEPHADEX® polymer, ceramic beads, cross-linked polyacrylamide beads, cubes, prills, or other gel forms, alginate beads, K-carrageenan cubes, as well as solid particles that can be recovered from the aqueous solutions due to inherent magnetic ability. The shape of the catalyst is variable (in that the desired dynamic properties of the particular entity are integrated with volume/surface area relationships that influence catalyst activity). Optionally, the induced microorganism is immobilized in alginate beads that have been cross-linked with polyethyleneimine or is immobilized in a polyacrylamide-type polymer.

Also provided are compositions that can be used in the disclosed methods, as well as for the production of various devices, such as biofilters. Optionally, the compositions comprise: (a) a nutrient medium comprising trehalose and, optionally, one or more amide containing amino acids, or derivatives thereof; (b) one or more enzyme-producing microorganisms; and (c) one or more enzymes. Optionally, the enzymes are selected from the group consisting of nitrile hydratase, amidase, asparaginase I, and combinations thereof. Optionally, the one or more microorganisms comprise bacteria selected from the group consisting of genus Pseudonocardia, genus Nocardia, genus Pseudomonas, genus Rhodococcus, genus Brevibacterium, and combinations thereof. By way of example, the microorganism is from the genus Rhodococcus. Optionally, the microorganism from the genus Rhodococcus is Rhodococcus rhodochrous DAP 96622, Rhodococcus sp. DAP 96523 or combinations thereof. Optionally, the microorganism is at least partially immobilized.

As described herein, the provided compositions and methods include the use of trehalose. The concentration of trehalose in the compositions or medium used in the provided methods can be at least 1 gram per liter (g/L). Optionally, the concentration of trehalose is in the range of 1 g/L to 50 g/L, or 1 g/L to 10 g/L. Optionally, the concentration of trehalose in the medium is at least 4 g/L.

Optionally, the compositions and medium used in the provided methods further comprise one or more amide containing amino acids or derivatives thereof. The amide containing amino acids can, for example, be selected from the group consisting of asparagine, glutamine, derivatives thereof, or combinations thereof. For example, the amide-containing amino acids may include natural forms of asparagines, anhydrous asparagine, asparagine monohydrate, natural forms of glutamine, anhydrous glutamine, and/or glutamine monohydrate, each in the form of the L-isomer or D-isomer.

The concentration of the amide containing amino acids or derivatives thereof in the medium can vary depending upon the desired end result of the culture. For example, a culture may be carried out for the purpose of producing microorganisms having a specific enzymatic activity. Optionally, a culture may be carried out for the purpose of forming and collecting a specific enzyme from the cultured microorganisms. Optionally, a culture may be carried out for the purpose of forming and collecting a plurality of enzymes having the same or different activities and functions.

The amount of the amide containing amino acids, or derivatives thereof, added to the growth medium or mixture can generally be up to 10,000 parts per million (ppm) (i.e., 1% by weight) based on the overall weight of the medium or mixture. The present methods are particularly beneficial, however, in that enzyme activity can be induced through addition of even lesser amounts. Optionally, the one or more amide containing amino acids are present at a concentration of at least 50 ppm. By way of other examples, the concentration of the amide containing amino acids or derivatives thereof is in the range of 50 ppm to 5,000 ppm, 100 ppm to 3,000 ppm, 200 ppm to 2,000 ppm, 250 ppm to 1500 ppm, 500 ppm to 1250 ppm, or 500 ppm to 1000 ppm.

Optionally, the trehalose and amide containing amino acids or derivatives thereof are added to a nutritionally complete media. A suitable nutritionally complete medium generally is a growth medium that can supply a microorganism with the necessary nutrients required for its growth, which minimally includes a carbon and/or nitrogen source. One specific example is the commercially available R2A agar medium, which typically consists of agar, yeast extract, proteose peptone, casein hydrolysate, glucose, soluble starch, sodium pyruvate, dipotassium hydrogenphosphate, and magnesium sulfate. Another example of a nutritionally complete liquid medium is Yeast Extract Malt Extract Agar (YEMEA), which consists of glucose, malt extract, and yeast extract (but specifically excludes agar). Any nutritionally complete medium known in the art could be used for the disclosed methods, the above media being described for exemplary purposes only. Such nutritionally complete media can be included in the compositions described herein.

Optionally, the disclosed compositions and media can contain further additives. Typically, the other supplements or nutrients are those useful for assisting in greater cell growth, greater cell mass, or accelerated growth. For example, the compositions and media can comprise a carbohydrate source in addition to any carbohydrate source already present in the nutritionally complete medium.

As described above, most media typically contain some content of carbohydrate (e.g., glucose); however, it can be useful to include an additional carbohydrate source. The type of excess carbohydrate provided can depend upon the desired outcome of the culture. For example, the addition of carbohydrates, such as maltose or maltodextrin, has been found to provide improved induction of asparaginase I and improved stability of nitrile hydratase.

Optionally, the compositions and media further comprise cobalt. Cobalt or a salt thereof can be added to the mixture or media. For example, the addition of cobalt (e.g., cobalt chloride) to the media can be particularly useful for increasing the mass of the enzyme produced by the cultured microorganisms. Cobalt or a salt thereof can, for example, be added to the culture medium such that the cobalt concentration is an amount up to 100 ppm. Cobalt can, for example, be present at a concentration of 5 ppm to 100 ppm, 10 ppm to 75 ppm, 10 ppm to 50 ppm, or 10 ppm to 25 ppm.

Optionally, the compositions and media further comprise urea. Urea or a salt thereof can be added to the mixture or media. Urea or a salt thereof can, for example, be added to the culture medium such that the urea concentration is in an amount up to 10 g/L. Urea can, for example, be present in a concentration of 5 g/L to 100 g/L, 10 g/L to 75 g/L, 10 g/L to 50 g/L, or 10 g/L to 25 g/L. Optionally, urea is present at a concentration of 7.5 g/L.

The compositions and media may also include further components. For example, other suitable medium components may include commercial additives, such as cottonseed protein, maltose, maltodextrin, and other commercial carbohydrates. Optionally, the medium further comprises maltose or maltodextrin. Maltose or maltodextrin, for example, can be added to the culture medium such that the maltose or maltodextrin concentration is at least 1 g/L. Optionally, maltose or maltodextrin can be present at a concentration of.

Optionally, the compositions and media are free of any nitrile containing compounds. Nitrile compounds were previously required in the culture medium to induce enzyme activity toward two or more nitrile compounds. The compositions described herein achieve this through the use of completely safe trehalose and/or amide containing amino acids or derivatives thereof; therefore, the medium can be free of any nitrile containing compounds.

A variety of microorganisms can be cultivated for use in the provided methods and compositions. Generally, any microorganism capable of producing enzymatic activity, as described herein, can be used. Optionally, the microorganisms are capable of producing nitrile hydratase.

As used herein, nitrile hydratase producing microorganisms are intended to refer to microorganisms that, while generally being recognized as being capable of producing nitrile hydratase, are also capable of producing one or more further enzymes. Further, most microorganisms are capable of producing a variety of enzymes, such production often being determined by the environment of the microorganism. Thus, while microorganisms for use herein may be disclosed as nitrile hydratase producing microorganisms, such language only refers to the known ability of such microorganisms to produce nitrile hydratase and does not limit the microorganisms to only the production of nitrile hydratase. On the contrary, a nitrile hydratase producing microorganism is a microorganism capable of producing at least nitrile hydratase (i.e., is capable of producing nitrile hydratase or nitrile hydratase and one or more further enzymes).

A number of nitrile hydratase producing microorganisms are known in the art. For example, bacteria belonging to the genus Nocardia [see Japanese Patent Application No. 54-129190], Rhodococcus [see Japanese Patent Application No. 2-470], Rhizobium [see Japanese Patent Application No. 5-236977], Klebsiella [Japanese Patent Application No. 5-30982], Aeromonas [Japanese Patent Application No. 5-30983], Agrobacterium [Japanese Patent Application No. 8-154691], Bacillus [Japanese Patent Application No. 8-187092], Pseudonocardia [Japanese Patent Application No. 8-56684], and Pseudomonas are non-limiting examples of nitrile hydratase producing microorganisms that can be used. Optionally, the nitrile hydratase producing microorganism comprises bacteria from the genus Rhodococcus.

Further, specific examples of microorganisms include, but are not limited to, Nocardia sp., Rhodococcus sp., Rhodococcus rhodochrous, Klebsiella sp., Aeromonas sp., Citrobacter freundii, Agrobacterium rhizogenes, Agrobacterium tumefaciens, Xanthobacter flavas, Erwinia nigrifluens, Enterobacter sp., Streptomyces sp., Rhizobium sp., Rhizobium loti, Rhizobium legminosarum, Rhizobium merioti, Candida guilliermondii, Pantoea agglomerans, Klebsiella pneumoniae subsp. pneumoniae, Agrobacterium radiobacter, Bacillus smithii, Pseudonocardia thermophila, Pseudomonas chloroaphis, Pseudomonas erythropolis, Brevibacterium ketoglutamicum, Rhodococcus erythropolis, and Pseudonocardia thermophila. Optionally, the microorganisms used can, for example, comprise Rhodococcus sp. DAP 96253 and DAP 96255 and Rhodococcus rhodochrous DAP 96622, and combinations thereof.

Optionally, the microorganisms can also include transformants. In particular, the transformants can be any host wherein a nitrile hydratase gene cloned from a microorganism known to include such a gene, is inserted and expressed. For example, U.S. Pat. No. 5,807,730 describes the use of Escherichia coli as a host for the MT-10822 bacteria strain (FERM BP-5785). Of course, other types of genetically engineered bacteria could be used herein so long as the bacteria are capable of producing one or more enzymes, as described herein.

Not all species within a given genus exhibit the same type of enzyme activity and/or production. Thus, it is possible to have a genus generally known to include strains capable of exhibiting a desired activity but have one or more species that do not generally exhibit the desired activity. Thus, host microorganisms can include strains of bacteria that are not specifically known to have the desired activity but are from a genus known to have specific strains capable of producing the desired activity. Such strains can have transferred thereto one or more genes useful to cause the desired activity. Non-limiting examples of such strains include Rhodococcus equi and Rhododoccus globerulus PWD1.

The microorganisms can be selected from known sources or can comprise newly isolated microorganisms. Optionally, microorganisms may be isolated and identified as useful microorganism strains by growing strains in the presence of trehalose and/or one or more amide containing amino acids or derivatives thereof. The microorganism can be isolated or selected or obtained from known sources or can be screened from future sources based on the ability to detoxify a mixture of nitriles or a mixture of nitrile and amide compounds or a mixture of amides to the corresponding amide and/or acid after multiple induction according to the present invention. Assays to determine whether the microorganism is useful are known in the art. For example, the presence of nitrile hydratase or amidase activity can be determined through detection of free ammonia. See Fawcett and Scott, “A Rapid and Precise Method for the Determination of Urea,” J. Clin. Pathol. 13:156-9 (1960), which is incorporated herein by reference.

The microorganisms can be cultured and harvested for achieving optimal biomass. In certain examples, such as when cultured on agar plates, the microorganisms can be cultured for a period of at least 24 hours but generally less than six days. When cultured in a fermentor, the microorganisms are preferably cultured in a minimal medium for a period of 1 hour to 48 hours, 1 hour to 20 hours, or 16 hours to 23 hours. If a larger biomass is desired, the microorganisms can be cultured in the fermentor for longer time periods. At the end of the culture period, the cultured microorganisms are typically collected and concentrated, for example, by scraping, centrifuging, filtering, or any other method known to those skilled in the art.

The microorganisms can be cultured under further specified conditions. For example, culturing is preferably carried out at a pH between 3.0 and 11.0, more preferably between 6.0 and 8.0. The temperature at which culturing is performed is preferably between 4° C. and 55° C., more preferably between 15° C. and 37° C. Further, the dissolved oxygen tension is preferentially between 0.1% and 100%, preferably between 4% and 80%, and more preferably between 4% and 30%. The dissolved oxygen tension may be monitored and maintained in the desired range by supplying oxygen in the form of ambient air, pure oxygen, peroxide, and, optionally, other compositions which liberate oxygen.

It is also possible according to the methods disclosed herein to separate the steps of microorganism growth and enzyme activity induction. For example, it is possible to grow one or more microorganisms on a first medium that does not include supplementation necessary to induce enzyme activity. Such first medium can be referred to as a growth phase medium for the microorganisms. In a second phase (i.e., an induction phase), the cultured microorganisms can be transferred to a second medium comprising supplementation necessary to induce enzyme activity. Such second medium would preferentially comprise the trehalose and/or one or more amide containing amino acids or derivatives thereof, as described herein.

Similarly, the induction supplements can be added at any time during cultivation of the desired microorganisms. For example, the media can be supplemented with trehalose and/or amide containing amino acids or derivatives thereof prior to beginning cultivation of the microorganisms. Alternately, the microorganisms could be cultivated on a medium for a predetermined amount of time to grow the microorganism, and trehalose and/or amide containing amino acids or derivatives thereof could be added at one or more predetermined times to induce the desired activity in the microorganisms. Moreover, the trehalose and/or amide containing amino acids or derivatives thereof could be added to the growth medium (or to a separate mixture including the previously grown microorganisms) to induce the desired activity in the microorganisms after the growth of the microorganisms is complete.

Provided are methods for detoxifying a mixture of nitriles by converting the nitriles to the corresponding amides or acids. Optionally, the method comprises applying a culture of nitrile degrading microorganisms to a mixture of nitriles and multiply inducing the microorganisms with a mixture of trehalose and/or amide containing amino acids or derivatives thereof for a sufficient amount of time to convert the nitriles to the corresponding amides. Alternatively, the method comprises applying multiply induced microorganisms to a mixture of nitriles for a sufficient amount of time to convert the nitriles to the corresponding amides.

When the microorganisms are applied to a waste stream, the microorganisms may be growing (actively dividing) or resting (not actively dividing). When the methods entail application of an actively growing culture of microorganisms, the application conditions are preferably such that bacterial growth is supported or sustained. When the methods entail application of a culture of microorganisms which are not actively dividing, the application conditions are preferably such that enzymatic activities are supported.

Optionally, the disclosed methods and compositions can be used to treat waste streams from a production plant having waste that typically contains high concentrations of nitriles, cyanohydrin(s), or other chemicals subject to enzymatic degradation. For example, provided are methods to detoxify a mixture of nitrile compounds or a mixture of nitrile and amide compounds in an aqueous waste stream from a nitrile production plant. Further, the present invention could be used for treatment of waste streams in the production of acrylonitrile butadiene styrene (ABS), wherein acrylonitrile is used in the production of the ABS.

Also provided is a biofilter that can be used in the detoxification of mixtures of nitrile compounds, mixtures of nitrile and amide compounds and mixtures of amide compounds in effluents such as air, vapors, aerosols, and water or aqueous solutions. For example, if volatile nitrile compounds are present, the volatiles may be stripped from solid or aqueous solution in which they are found and steps should be carried out in such a way that the volatiles are trapped in a biofilter. Once trapped, the volatiles can be detoxified with a pure culture or an extract of a microorganism, as described herein.

Further provided are kits comprising a culture of a microorganism which has been multiply induced and is able to detoxify a mixture of nitrile compounds, a mixture of nitrile and amide compounds, or a mixture of amide compounds. The microorganism can be actively dividing or lyophilized and can be added directly to an aqueous solution containing the nitrile and/or amide compounds. Optionally, the kit comprises an induced lyophilized sample. The microorganism also can be immobilized onto a solid support, as described herein. Other kit components can include, for example, a mixture of induction supplements, as described herein, for induction of the microorganisms, as well as other kit components, such as vials, packaging components, and the like, which are known to those skilled in the art.

Also provided are methods for delaying a plant development process comprising exposing a plant or plant part to one or more enzymes or a microorganism producing the enzymes. Optionally, the microorganisms used to delay the plant development process are treated with an inducing and/or stabilization agent as described herein, including, for example, trehalose, amide containing amino acids, cobalt, urea, and mixtures thereof. By way of example, provided is a method of delaying a plant development process comprising exposing a plant or plant part to one or more enzymes, wherein the enzymes are produced by one or more bacteria by culturing the bacteria in a medium comprising trehalose and, optionally, one or more amid containing amino acids, and wherein the enzymes are exposed to the plant or plant part in a quantity sufficient to delay the plant development process.

Optionally, the methods are drawn to delaying a plant development process comprising exposing a plant or plant part to one or more bacteria selected from the group consisting of Rhodococcus spp., Pseudomonas chloroaphis, Brevibacterium ketoglutamicum, and mixtures thereof. The one or more bacteria are cultured in a medium comprising trehalose and, optionally, one or more amide containing amino acids or derivatives thereof and exposed to the plant or plant part in a quantity sufficient to delay the plant development process. The provided methods may be used, for example, to delay fruit/vegetable ripening or flower senescence and to increase the shelf-life of fruit, vegetables, or flowers, thereby facilitating transportation, distribution, and marketing of such plant products. Methods for delaying a plant development process are described in U.S. Publication No. 2008/0236038, which is incorporated herein by reference.

Optionally, the method comprises exposing a plant or plant part to one or more enzymes or an extract from the bacteria. The enzyme or extract is exposed to the plant or plant part in a quantity sufficient to delay the plant development process. For example, provided is a method for delaying a plant development process comprising exposing a plant or plant part to an enzymatic extract of one or more bacteria, wherein the bacteria are cultured in a medium comprising trehalose and one or more amide containing amino acids, and wherein the enzymatic extract is exposed to the plant or plant part in a quantity sufficient to delay the plant development process.

As used herein, exposing a plant or plant part to one or more of the above bacteria includes, for example, exposure to intact bacterial cells, bacterial cell lysates, and bacterial extracts that possess enzymatic activity (i.e., “enzymatic extracts”). Methods for preparing lysates and enzymatic extracts from cells, including bacterial cells, are known. The one or more bacteria used in the methods provided may at times be more generally referred to herein as the “catalyst.”

As used herein, “plant” or “plant part” is broadly defined to include intact plants and any part of a plant, including but not limited to fruit, vegetables, flowers, seeds, leaves, nuts, embryos, pollen, ovules, branches, kernels, ears, cobs, husks, stalks, roots, root tips, anthers, and the like. The plant part can, for example, be a fruit, a vegetable, or a flower. Optionally, the plant part is a fruit, more particularly a climacteric fruit, as described in more detail below.

The disclosed methods are directed to delaying a plant development process, such as a plant development process generally associated with increased ethylene biosynthesis. “Plant development process” is intended to mean any growth or development process of a plant or plant part, including but not limited to fruit ripening, vegetable ripening, flower senescence, leaf abscission, seed germination, and the like. Optionally, the plant development process is fruit or vegetable ripening, flower senescence, or leaf abscission, more particularly fruit or vegetable ripening. As defined herein, “delaying a plant development process,” and grammatical variants thereof, refers to any slowing, interruption, suppression, or inhibition of the plant development process of interest or the phenotypic or genotypic changes to the plant or plant part typically associated with the specific plant development process. For example, when the plant development process is fruit ripening, a delay in fruit ripening may include inhibition of the changes generally associated with the ripening process (e.g., color change, softening of pericarp (i.e., ovary wall), increases in sugar content, changes in flavor, general degradation/deterioration of the fruit, and eventual decreases in the desirability of the fruit to consumers, as described above). One of skill in the art will appreciate that the length of time required for fruit ripening to occur will vary depending on, for example, the type of fruit and the specific storage conditions utilized (e.g., temperature, humidity, air flow, etc.). Accordingly, “delaying fruit ripening” may constitute a delay of 1 to 90 days, particularly 1 to 30 days, more particularly 5 to 30 days. Methods for assessing a delay in a plant development process such as fruit ripening, vegetable ripening, flower senescence, and leaf abscission are well within the routine capabilities of those of ordinary skill in the art and may be based on, for example, comparison to plant development processes in untreated plants or plant parts. Optionally, delays in a plant development process resulting from the disclosed methods may be assessed relative to untreated plants or plant parts or to plants or plant parts that have been treated with one or more agents known to retard the plant development process. For example, a delay in fruit ripening resulting from the provided methods may be compared to fruit ripening times of untreated fruit or fruit that has been treated with an anti-ripening agent, such as those described herein.

The one or more bacteria are exposed to the plant or plant part in a quantity sufficient to delay the plant development process. “Exposing” a plant or plant part to one or more of the bacteria includes any method for presenting a bacterium to the plant or plant part. Indirect methods of exposure include, for example, placing the bacterium or mixture of bacteria in the general proximity of the plant or plant part (i.e., indirect exposure). Optionally, the bacteria may be exposed to the plant or plant part via closer or direct contact. Furthermore, as defined herein, a “sufficient” quantity of the one or more bacteria of the invention will depend on a variety of factors, including but not limited to, the particular bacteria utilized in the method, the form in which the bacteria is exposed to the plant or plant part (e.g., as intact bacterial cells, cell lysates, or enzymatic extracts, as described above), the means by which the bacteria is exposed to the plant or plant part, and the length of time of exposure. Those of skill in the art can determine the “sufficient” quantity of the one or more bacteria necessary to delay the plant development process through routine experimentation.

The one or more bacteria are “induced” to exhibit a desired characteristic (e.g., the ability to delay a plant development process such as fruit ripening) by exposure to or treatment with a suitable inducing agent. Inducing agents include but are not limited to trehalose, asparagine, glutamine, cobalt, urea, or any mixture thereof. Optionally, the bacteria are exposed to or treated with the inducing agent asparagine, more particularly a mixture of the inducing agents comprising trehalose, asparagine, cobalt, and urea. The inducing agent can be added at any time during cultivation of the desired cells.

While not intending to be limited to a particular mechanism, “inducing” the bacteria may result in the production (or increased production) of one or more enzymes, as described above, such as a nitrile hydratase, amidase, and/or asparaginase, and the induction of one or more of these enzymes may play a role in delaying a plant development process of interest. “Nitrile hydratases,” “amidases,” and “asparaginases” comprise families of enzymes present in cells from various organisms, including but not limited to, bacteria, fungi, plants, and animals. Such enzymes are well known, and each class of enzyme possesses recognized enzymatic activities.

Methods of delaying a plant development process comprising exposing a plant or plant part to one or more enzymes selected from the group consisting of nitrile hydratase, amidase, asparaginase, or a mixture thereof, wherein the one or more enzymes are exposed to the plant or plant part in a quantity or at an enzymatic activity level sufficient to delay the plant development process. For example, whole cells that produce, are induced to produce, or are genetically modified to produce one or more of the above enzymes (i.e., nitrile hydratase, amidase, and/or asparaginase) may be used in methods to delay a plant development process. Alternatively, the nitrile hydratase, amidase, and/or asparaginase may be isolated, purified, or semi-purified from any the above cells and exposed to the plant or plant part in a more isolated form. See, for example, Goda et al., J. Biol. Chem. 276:23480-5 (2001); Nagasawa et al., Eur. J. Biochem. 267:138-144 (2000); Soong et al., Appl. Environ. Microbiol. 66:1947-52 (2000); Kato et al., Eur. J. Biochem. 263:662-70 (1999), all of which are herein incorporated by reference in their entirety. Optionally, a single cell type may be capable of producing (or being induced or genetically modified to produce) more than one of the enzymes. Such cells are suitable for use in the disclosed methods.

The disclosed methods may be used to delay a plant development process of any plant or plant part. Optionally, the methods are directed to delaying ripening and the plant part is a fruit (climacteric or non-climacteric), vegetable, or other plant part subject to ripening. One of skill in the art will recognize that “climacteric fruits” exhibit a sudden burst of ethylene production during fruit ripening, whereas “nonclimacteric fruits” are generally not believed to experience a significant increase in ethylene biosynthesis during the ripening process. Exemplary fruits, vegetables, and other plant products include but are not limited to: apples, apricots, biriba, breadfruit, cherimoya, feijoa, fig, guava, jackfruit, kiwi, bananas, peaches, avocados, apples, cantaloupes, mangos, muskmelons, nectarines, persimmon, sapote, soursop, olives, papaya, passion fruit, pears, plums, tomatoes, bell peppers, blueberries, cacao, caju, cucumbers, grapefruit, lemons, limes, peppers, cherries, oranges, grapes, pineapples, strawberries, watermelons, tamarillos, and nuts.

Optionally, the methods are drawn to delaying flower senescence, wilting, abscission, or petal closure. Any flower may be used herein. Exemplary flowers include but are not limited to roses, carnations, orchids, portulaca, malva, and begonias. Cut flowers, more particularly commercially important cut flowers such as roses and carnations, are of particular interest. Optionally, flowers that are sensitive to ethylene are used herein. Ethylene-sensitive flowers include but are not limited to flowers from the genera Alstroemeria, Aneomone, Anthurium, Antirrhinum, Aster, Astilbe, Cattleya. Cymbidium, Dahlia, Dendrobium, Dianthus, Eustoma, Freesia, Gerbera, Gypsophila, Iris, Lathyrus, Lilium, Limonium, Nerine, Rosa, Syringa, Tulipa, and Zinnia Representative ethylene-sensitive flowers also include those of the families Amarylidaceae, Alliaceae, Convallariaceae, Hemerocallidaceae, Hyacinthaceae, Liliaceae, Orchidaceae, Aizoaceae, Cactaceae, Campanulaceae, Caryophyllaceae, Crassulaceae, Gentianaceae, Malvaceae, Plumbaginaceae, Portulacaceae, Solanaceae, Agavacaea, Asphodelaceae, Asparagaceae, Begoniaceae, Caprifoliaceae, Dipsacaceae, Euphorbiaceae, Fabaceae, Lamiaceae, Myrtaceae, Onagraceae, Saxifragaceae, and Verbenaceae. See, for example, Van Doom, Annals of Botany 89:375-383 (2002); Van Doom, Annals of Botany 89:689-693 (2002); and Elgar, “Cut Flowers and Foliage—Cooling Requirements and Temperature Management” at hortnet.co.nz/publications/hortfacts/hf305004.htm (1998) (last accessed Mar. 20, 2007), all of which are herein incorporated by reference in their entirety. Methods for delaying leaf abscission are also disclosed herein. Significant commercial interest exists in the plant, fruit, vegetable, and flower industries for methods for regulating plant development processes such as ripening, senescence, and abscission.

The skilled artisan will further recognize that any of the methods disclosed herein can be combined with other known methods for delaying a plant development process, particularly those processes generally associated with increased ethylene biosynthesis (e.g., fruit/vegetable ripening, flower senescence, and leaf abscission). Moreover, as described above, increased ethylene production has also been observed during attack of plants or plant parts by pathogenic organisms. Accordingly, the methods may find further use in improving plant response to pathogens.

Generally, any bacterial, fungal, plant, or animal cell capable of producing or being induced to produce nitrile hydratase, amidase, asparaginase, or any combination thereof may be used herein. A nitrile hydratase, amidase, and/or asparaginase may be produced constitutively in a cell from a particular organism (e.g., a bacterium, fungus, plant cell, or animal cell) or, alternatively, a cell may produce the desired enzyme or enzymes only following “induction” with a suitable inducing agent. “Constitutively” is intended to mean that at least one enzyme of the invention is continually produced or expressed in a particular cell type. Other cell types, however, may need to be “induced,” as described above, to express nitrile hydratase, amidase, and/or asparaginase at a sufficient quantity or enzymatic activity level to delay a plant development process of interest. That is, an enzyme of the invention may only be produced (or produced at sufficient levels) following exposure to or treatment with a suitable inducing agent. Such inducing agents are known and outlined above. For example, the one or more bacteria are treated with an inducing agent such as asparagine, glutamine, cobalt, urea, or any mixture thereof, more particularly a mixture of asparagine, cobalt, and urea. Furthermore, as disclosed in U.S. Pat. Nos. 7,531,343 and 7,531,344, which are incorporated by reference in their entireties, entitled “Induction and Stabilization of Enzymatic Activity in Microorganisms,” asparaginase I activity can be induced in Rhodococcus rhodochrous DAP 96622 (Gram-positive) or Rhodococcus sp. DAP 96253 (Gram-positive), in medium supplemented with amide containing amino acids or derivatives thereof. Other strains of Rhodococcus can also preferentially be induced to exhibit asparaginase I enzymatic activity utilizing amide containing amino acids or derivatives thereof.

P. chloroaphis (ATCC Deposit No. 43051), which produces asparaginase I activity in the presence of asparagine, and B. kletoglutamicum (ATCC Deposit No. 21533), a Gram-positive bacterium that has also been shown to produce asparaginase activity, are also used in the disclosed methods. Fungal cells, such as those from the genus Fusarium, plant cells, and animal cells, that express a nitrile hydratase, amidase, and/or an asparaginase, may also be used herein, either as whole cells or as a source from which to isolated one or more of the above enzymes.

The nucleotide and amino acid sequences for several nitrile hydratases, amidases, and asparaginases from various organisms are disclosed in publicly available sequence databases. A non-limiting list of representative nitrile hydratases and aliphatic amidases known in the art is set forth in Tables 1 and 2 and in the sequence listing. The “protein score” referred to in Tables 1 and 2 provides an overview of percentage confidence intervals (% Confid. Interval) of the identification of the isolated proteins based on mass spectroscopy data.

TABLE 1
Amino Acid Sequence Information for Representative Nitrile Hydratases

| | | | Protein Score |
|---|---|---|---|
| | Accession | | (% Confid. |
| Source organism | No. | Sequence Identifier | Interval) |
| Rhodococcus sp. | 806580 | SEQ ID NO: 1 | 100% |
| Nocardia sp. | 27261874 | SEQ ID NO: 2 | 100% |
| Rhodococcus | 49058 | SEQ ID NO: 3 | 100% |
| rhodochrous | | | |
| Uncultured bacterium | 27657379 | SEQ ID NO: 4 | 100% |
| (BD2); beta-subunit of | | | |
| nitrile hydratase | | | |
| Rhodococcus sp. | 806581 | SEQ ID NO: 5 | 100% |
| Rhodococcus | 581528 | SEQ ID NO: 6 | 100% |
| rhodochrous | | | |
| Uncultured bacterium | 7657369 | SEQ ID NO: 7 | 100% |
| (SP1); alpha-subunit of | | | |
| nitrile hydratase | | | |

TABLE 2
Amino Acid Sequence Information for Representative Aliphatic Amidases

| | | | Protein |
|---|---|---|---|
| | | | Score |
| | Accession | Sequence | (% Confid. |
| Source organism | No. | Identifier | Interval) |
| Rhodococcus rhodochrous | 62461692 | SEQ ID NO: 8 | 100% |
| Nocardia farcinica IFM | 54022723 | SEQ ID NO: 9 | 100% |
| 10152 | | | |
| Pseudomonas aeruginosa | 15598562 | SEQ ID NO: 10 | 98.3% |
| PAO1 | | | |
| Helicobacter pylori J99 | 15611349 | SEQ ID NO: 11 | 99.6% |
| Helicobacter pylori 26695 | 2313392 | SEQ ID NO: 12 | 97.7% |
| Pseudomonas aeruginosa | 150980 | SEQ ID NO: 13 | 94% |

Optionally, host cells that have been genetically engineered to express a nitrile hydratase, amidase, and/or asparaginase can be exposed to a plant or plant part for delaying a plant development process. Specifically, a polynucleotide that encodes a nitrile hydratase, amidase, or asparaginase (or multiple polynucleotides each of which encodes a nitrile hydratase, amidase, or asparaginase) may be introduced by standard molecular biology techniques into a host cell to produce a transgenic cell that expresses one or more of the enzymes. The use of the terms “polynucleotide,” “polynucleotide construct,” “nucleotide,” or “nucleotide construct” is not intended to limit to polynucleotides or nucleotides comprising DNA. Those of ordinary skill in the art will recognize that polynucleotides and nucleotides can comprise ribonucleotides and combinations of ribonucleotides and deoxyribonucleotides. Such deoxyribonucleotides and ribonucleotides include both naturally occurring molecules and synthetic analogues. The polynucleotides described herein encompass all forms of sequences including, but not limited to, single-stranded forms, double-stranded forms, and the like.

Variants and fragments of polynucleotides that encode polypeptides that retain the desired enzymatic activity (i.e., nitrile hydratase, amidase, or asparaginase activity) may also be used herein. By “fragment” is intended a portion of the polynucleotide and hence also encodes a portion of the corresponding protein. Polynucleotides that are fragments of an enzyme nucleotide sequence generally comprise at least 10, 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length enzyme polynucleotide sequence. A polynucleotide fragment will encode a polypeptide with a desired enzymatic activity and will generally encode at least 15, 25, 30, 50, 100, 150, 200, or 250 contiguous amino acids, or up to the total number of amino acids present in a full-length enzyme amino acid sequence. “Variant” is intended to mean substantially similar sequences. Generally, variants of a particular enzyme sequence will have at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the reference enzyme sequence, as determined by standard sequence alignment programs. Variant polynucleotides described herein will encode polypeptides with the desired enzyme activity.

As used in the context of production of transgenic cells, the term “introducing” is intended to mean presenting to a host cell, particularly a microorganism such as Escherichia coli, with a polynucleotide that encodes a nitrile hydratase, amidase, and, optionally, asparaginase. Optionally, the polynucleotide will be presented in such a manner that the sequence gains access to the interior of a host cell, including its potential insertion into the genome of the host cell. The disclosed methods do not depend on a particular protocol for introducing a sequence into a host cell, only that the polynucleotide gains access to the interior of at least one host cell. Methods for introducing polynucleotides into host cells are well known, including, but not limited to, stable transfection methods, transient transfection methods, and virus-mediated methods. “Stable transfection” is intended to mean that the polynucleotide construct introduced into a host cell integrates into the genome of the host and is capable of being inherited by the progeny thereof. “Transient transfection” or “transient expression” is intended to mean that a polynucleotide is introduced into the host cell but does not integrate into the host's genome.

Furthermore, the nitrile hydratase, amidase, or asparaginase nucleotide sequence may be contained in, for example, a plasmid for introduction into the host cell. Typical plasmids of interest include vectors having defined cloning sites, origins of replication, and selectable markers. The plasmid may further include transcription and translation initiation sequences and transcription and translation terminators. Plasmids can also include generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or optimally both. For general descriptions of cloning, packaging, and expression systems and methods, see Giliman and Smith, Gene 8:81-97 (1979); Roberts et al., Nature 328:731-734 (1987); Berger and Kimmel, Guide to Molecular Cloning Techniques, Methods in Enzymology, Vol. 152 (Academic Press, Inc., San Diego, Calif.) (1989); Sambrook et al., Molecular Cloning: A Laboratory Manual, Vols. 1-3 (2d ed; Cold Spring Harbor Laboratory Press, Plainview, N.Y.) (1989); and Ausubel et al., Current Protocols in Molecular Biology, Current Protocols (Greene Publishing Associates, Inc., and John Wiley & Sons, Inc., New York; 1994 Supplement) (1994). Transgenic host cells that express one or more of the enzymes may be used in the disclosed methods as whole cells or as a biological source from which one or more enzymes can be isolated.

### EXAMPLES

### Example 1

### Nitrile Hydratase and Amidase Induction

The induction of nitrile hydratase activity and amidase activity in Rhodococcus sp., strain DAP 96253, was evaluated using multiple types of inducers (1000 ppm). Three different samples were cultured in YEMEA medium containing 10 ppm cobalt and 7.5 g/L urea and supplemented with acrylonitrile, asparagine, or glutamine. The specific nitrile hydratase activity and the specific amidase activity in each sample was evaluated, and the results are provided below in Table 3, with activities provided in units/mg cdw (cell dry weight). One unit of nitrile hydratase activity relates to the ability to convert 1 μmol of acrylonitrile to its corresponding amide per minute, per milligram of cells (dry weight) at a pH of 7.0 and a temperature of 30° C. One unit of amidase activity relates to the ability to convert 1 μmol of acrylamide to its corresponding acid per minute, per milligram of cells (dry weight) pH of 7.0 and a temperature of 30° C.

TABLE 3

| | Nitrile Hydratase Activity | Amidase Activity |
|---|---|---|
| Supplement | (Units/mg cdw) | (Units/mg cdw) |
| Acrylonitrile | 162.23 | 7.59 |
| Asparagine | 170.50 | 13.24 |
| Glutamine | 173.45 | 10.39 |

As seen in Table 3, the use of asparagine or glutamine as an inducer for nitrile hydratase activity exceeds the ability of acrylonitrile to induce such activity. Moreover, the use of glutamine as an inducer resulted in amidase activity approximately 37% greater than the amidase activity resulting from the use of acrylonitrile, and asparagine provided approximately 74% greater activity than acrylonitrile.

### Example 2

### Stabilization of Nitrile Hydratase Activity Using Calcium Alginate Immobilization

Testing was performed to evaluate the relative stability of cells induced for nitrile hydratase activity using asparagine in the culture medium. Rhodococcus sp., strain DAP 96253, was cultured using a standard culture medium alone or supplemented with asparagine. Cells were recovered from the culture and immobilized in calcium alginate beads (2-3 mm diameter). To prepare the beads, 25 grams (g) of a 4% sodium alginate solution (1 g sodium alginate in 24 milliliters (ml) of 5 mM TRIS-HCl—pH 7.2) was prepared, and 25 milligrams of sodium meta-periodate was dissolved therein (stirred at 25° C. for 1 hour or until all alginate has dissolved). The cells for immobilization were suspended in 50 mM TRIS-HCl to a final volume of 50 ml, and the cell solution was added to the alginate mixture while stirring. Beads were formed by extruding the mixture through a 27G hypodermic needle into 500 ml of 0.1M CaCl₂. The beads were cured for 1 hour in the CaCl₂ solution and washed with water.

Four samples were prepared for evaluation: Sample 1—beads formed with cells cultured without asparagine but with asparagine added to the mixture including the beads; Sample 2—beads formed with cells cultured with asparagine and having asparagine added to the mixture including the beads; Sample 3—beads formed with cells cultured with asparagine and having a mixture of acrylonitrile and acetonitrile added to the mixture including the beads; and Sample 4—beads formed with cells cultured with acrylonitrile and acetonitrile and having asparagine added to the mixture including the beads. In samples 3 and 4, acrylonitrile and acetonitrile were added in a concentration of 500 parts per million (ppm) each. In each of samples 1-4, asparagine was added at 1000 ppm.

The immobilized cells were maintained for a time of about 150 hours and periodically evaluated for the remaining nitrile hydratase activity. The results of the test are illustrated in FIG. 1. For evaluation of stabilized activity, equivalent amounts of cells were tested, and the activity of an equivalent aliquot of whole cells at time 0 was set as 100%. Equivalent aliquots of catalyst were incubated at the appropriate temperature. At the appropriate times, an entire aliquot was removed from incubation and the enzyme activity determined. For the first 10 hours samples were evaluated every 2 hours. From 10-60 hours samples were evaluated every 4 hours and thereafter, samples were evaluated every 12 hours.

As seen in FIG. 1, immobilization of induced cells in calcium alginate provides stabilization of nitrile hydratase activity that is very similar to the level of stabilization achievable using hazardous nitrile containing compounds but without the disadvantages (e.g., health and regulatory issues).

### Example 3

### Stabilization of Nitrile Hydratase Activity Using Polyacrylamide Immobilization

Testing was performed to evaluate the relative stability of cells induced for nitrile hydratase activity using asparagine in the culture medium. Rhodococcus sp., strain DAP 96253, was cultured using a standard culture medium supplemented with asparagine. Cells were recovered from the culture and immobilized in cross-linked polyacrylamide cubes (2.5 mm×2.5 mm×1 mm). The polyacrylamide solution was prepared, and the desired loading of cells was added. The polyacrylamide with the cells was cross-linked to form a gel, which was cut into the noted cubes. No further known stabilizers were added to the polyacrylamide. Two samples were prepared for evaluation: Sample 1—cubes with low cell load (prepared with suspension comprising 1 g of cells per 40 mL of cell suspension); and Sample 2—cubes with high cell load (prepared with suspension comprising 4 g of cells per 40 mL of cell suspension).

The immobilized cells were maintained for a time of about 150 days and periodically evaluated for the remaining nitrile hydratase activity. The results of the test are illustrated in FIG. 2. For evaluation of stabilized activity, equivalent amounts of cells were tested, and the activity of an equivalent aliquot of whole cells at time 0 was set as 100%. Equivalent aliquots of catalyst were incubated at the appropriate temperature. At the appropriate times, an entire aliquot was removed from incubation and the enzyme activity determined. For the first 10 hours samples were evaluated every 2 hours. From 10-60 hours samples were evaluated every 4 hours. From 5 days to 40 days samples were evaluated every 12 hours. From 40 to 576 days, samples were evaluated on average every 10 days.

As seen in FIG. 2, cells stabilized using polyacrylamide maintained activity as much as 150 hours after induction. Moreover, polyacrylamide-immobilized cells loaded at a low concentration still exhibited 50% of the initial activity at about 45 hours after induction, and polyacrylamide-immobilized cells loaded at a high concentration still exhibited 50% of the initial activity at about 80 hours after induction.

### Example 4

### Stabilization of Nitrile Hydratase Activity Using Calcium Alginate or Polyacrylamide Immobilization

Testing was performed to evaluate the relative stability of cells induced for nitrile hydratase activity using asparagine in the culture medium. The testing specifically compared the stabilization provided by immobilization in polyacrylamide or calcium alginate. Rhodococcus sp., strain DAP 96622, was cultured using a standard culture medium supplemented with asparagine to induce nitrile hydratase activity. Cells were recovered from the culture for immobilization.

Test Sample 1 was prepared by immobilizing the asparagine induced cells in polyacrylamide cubes (2.5 mm×2.5 mm×1 mm) using the method described in Example 3. As a comparative, cells separately induced using acrylonitrile were also immobilized in polyacrylamide cubes for evaluation.

Test Sample 2 was prepared by immobilizing the asparagine induced cells in calcium alginate beads (2-3 mm diameter) using the method described in Example 2. As a comparative example, one sample was prepared using actual nitrile containing waste water as the inducing supplement (denoted NSB/WWCB). A second comparative example was prepared using, as the inducer, a synthetic mixture containing the dominant nitriles and amides present in an acrylonitrile production waste stream (also including ammonium sulfate and expressly excluding hydrogen cyanide) (denoted FC w/ AMS w/o HCN).

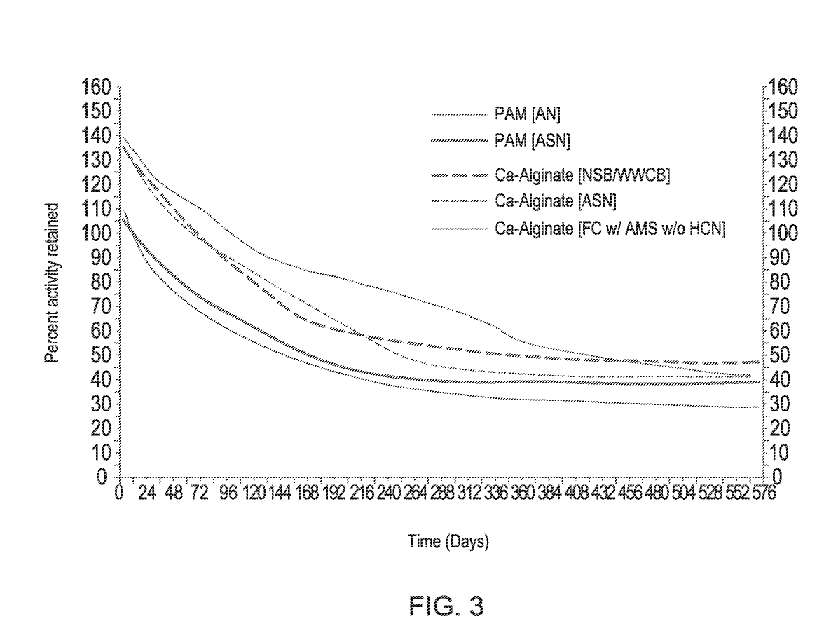

The immobilized cells were maintained for a time of about 576 days and periodically evaluated for the remaining nitrile hydratase activity. The results of the test are illustrated in FIG. 3. For evaluation of stabilized activity, equivalent amounts of cells were tested. The activity of an equivalent aliquot of whole cells at time 0 was set as 100%. Equivalent aliquots of catalyst were incubated at the appropriate temperature. At the appropriate times, an entire aliquot was removed from incubation and the enzyme activity determined. For the first 10 hours samples were evaluated every 2 hours. From 10-60 hours samples were evaluated every 4 hours. From 5 days to 40 days samples were evaluated every 12 hours. From 40 to 576 days, samples were evaluated on average every 10 days.

### Example 5

### Stabilization of Nitrile Hydratase Activity Using Glutaraldehyde Immobilization

Testing was performed to evaluate the relative stability of cells induced for nitrile hydratase activity using asparagine in the culture medium. The testing specifically compared the stabilization provided by immobilization via glutaraldehyde cross-linking Rhodococcus sp., strain DAP 96253, and Rhodococcus rhodochrous, strain DAP 96622, were separately cultured using a standard culture medium supplemented with asparagine to induce nitrile hydratase activity. Cells were recovered from the culture and cross-linked using glutaraldehyde, as described herein.

The immobilized cells were maintained for a time of about 576 days and periodically evaluated for the remaining nitrile hydratase activity. The results of the test are illustrated in FIG. 4. For evaluation of stabilized activity, equivalent amounts of cells were tested. The activity of an equivalent aliquot of whole cells at time 0 was set as 100%. Equivalent aliquots of catalyst were incubated at the appropriate temperature. At the appropriate times, an entire aliquot was removed from incubation and the enzyme activity determined. For the first 10 hours samples were evaluated every 2 hours. From 10-60 hours samples were evaluated every 4 hours. From 5 days to 40 days samples were evaluated every 12 hours. From 40 to 576 days, samples were evaluated on average every 10 days.

As seen in FIG. 4, both strains immobilized via glutaraldehyde cross-linking exhibited somewhat less initial activity in comparison to other stabilizations methods described above. However, both strains immobilized via glutaraldehyde cross-linking exhibited excellent long-term stabilization maintaining as much as 65% activity after 576 days.

### Example 6

### Effect of Asparagine and Glutamine on Growth of Nitrile Hydratase Producing Microorganisms

The relative growth of various nitrile hydratase producing microorganisms was evaluated. All strains were grown on YEMEA medium containing 7.5 g/L of urea and 10 ppm cobalt (provided as cobalt chloride) supplemented with asparagine (ASN), glutamine (GLN), or both asparagine and glutamine. The asparagine and glutamine were added at a concentration of 3.8 mM. Growth temperature was in the range of 26° C. to 30° C. Growth was evaluated by visual inspection and graded on the following scale: (−) meaning no detectable growth; (+/−) meaning scant growth; (+) meaning little growth; (++) meaning good growth; (+++) meaning very good growth; and (++++) meaning excellent growth. The results are provided below in Table 4.

TABLE 4

| | | | Growth Medium | | |
|---|---|---|---|---|---|
| | | Growth | Supplementation | | |
| | | Temp. | | | ASN + |
| Strain | ATCC # | (° C.) | ASN | GLN | GLN |
| Pseudomonas chloroaphis | 43051 | 30 | + | − | + |
| Pseudomonas chloroaphis | 13985 | 26 | + | + | ++ |
| Brevibacterium | 21533 | 30 | + | + | + |
| ketoglutaricum | | | | | |
| Rhodococcus erythropolis | 47072 | 26 | ++ | ++ | +++ |
| Rhodococcus sp. DAP | 55899 | 30 | ++++ | ++++ | ++++ |
| 96253 | | | | | |
| Rhodococcus rhodochrous | 55898 | 26 | ++++ | ++++ | ++++ |
| DAP 96622 | | | | | |

### Example 7

### Effect of Asparagine and Glutamine on Nitrile Hydratase and Amidase Production

The induction of nitrile hydratase production and amidase production in various nitrile hydratase producing microorganisms was evaluated. All strains were grown on YEMEA medium containing 7.5 g/L of urea and 10 ppm cobalt (provided as cobalt chloride) supplemented with asparagine (ASN), glutamine (GLN), or both asparagine and glutamine. The asparagine and glutamine were added at a concentration of 3.8 mM. As a comparative, enzyme production with no supplementation was also tested. Growth temperature was in the range of 26° C. to 30° C. The nitrile hydratase level in Units per mg of cell dry weight was evaluated, and the results are provided in Table 5. The amidase level in units per mg of cell dry weight was evaluated, and the results are provided in Table 6.

TABLE 5

| | | | Nitrile Hydratase Level | | | |
|---|---|---|---|---|---|---|
| | | Growth | (Units/mg cdw) Based on | | | |
| | ATCC | Temp. | Growth Medium Supplementation | | | |
| Strain | # | (° C.) | ASN | GLN | ASN + GLN | None |
| Pseudomonas | 43051 | 30 | 28 | No | 45 | 49 |
| chloroaphis | | | | growth | | |
| Pseudomonas | 13985 | 26 | 14 | 0 | 8 | 30 |
| chloroaphis | | | | | | |
| Brevibacterium | 21533 | 30 | 30 | 37 | 42 | 34 |
| ketoglutaricum | | | | | | |
| Rhodococcus | 47072 | 26 | 48 | 42 | 55 | 55 |
| erythropolis | | | | | | |
| Rhodococcus | 55899 | 30 | 155 | 135 | 152 | 82 |
| sp. DAP 96253 | | | | | | |
| Rhodococcus | 55898 | 26 | 158 | 160 | 170 | 63 |
| rhodochrous | | | | | | |
| DAP 96622 | | | | | | |

TABLE 6

| | | | Amidase Level (Units/mg | | | |
|---|---|---|---|---|---|---|
| | | Growth | cdw) Based on Growth | | | |
| | ATCC | Temp. | Medium Supplementation | | | |
| Strain | # | (° C.) | ASN | GLN | ASN + GLN | None |
| Pseudomonas | 43051 | 30 | 0 | No | 0 | 0 |
| chloroaphis | | | | growth | | |
| Pseudomonas | 13985 | 26 | 14 | 0 | 8 | 4 |
| chloroaphis | | | | | | |
| Brevibacterium | 21533 | 30 | 0 | 0 | 3 | 2 |
| ketoglutaricum | | | | | | |
| Rhodococcus | 47072 | 26 | 9 | 14 | 6 | 2 |
| erythropolis | | | | | | |
| Rhodococcus | 55899 | 30 | 13 | 7 | 10 | 4 |
| sp. DAP 96253 | | | | | | |
| Rhodococcus | 55898 | 26 | 10 | 6 | 12 | 5 |
| rhodochrous | | | | | | |
| DAP 96622 | | | | | | |

### Example 8

### Effect of Asparagine and Glutamine on Asparaginase I Production

The induction of asparaginase I production in various nitrile hydratase producing microorganisms was evaluated. All strains were grown on YEMEA medium containing 7.5 g/L of urea and 10 ppm cobalt (provided as cobalt chloride) supplemented with asparagine (ASN), glutamine (GLN), or both asparagine and glutamine. The asparagine and glutamine were added at a concentration of 3.8 mM. As a comparative, enzyme production was also evaluated with supplementation with acrylonitrile (AN), acrylamide (AMD) or acrylonitrile and acrylamide. Growth temperature was in the range of 26° C. to 30° C. The asparaginase I level in units per mg of cell dry weight was evaluated, and the results are provided in Table 7.

TABLE 7

| | | | Asparaginase I Level (Units/mg cdw) Based | | | | | |
|---|---|---|---|---|---|---|---|---|
| | | Growth | on Growth Medium Supplementation | | | | | |
| | | Temp. | | | AN/ | | | ASN/ |
| Strain | ATCC # | (° C.) | AN | AMD | AMD | ASN | GLN | GLN |
| Pseudomonas | 43051 | 30 | — | — | — | 18.4 | No | 18.7 |
| chloroaphis | | | | | | | Growth | |
| Pseudomonas | 13985 | 26 | 2 | 0 | 3 | 0 | 0 | 1 |
| chloroaphis | | | | | | | | |
| Brevibacterium | 21533 | 30 | 14.6 | 15.4 | 13.6 | 19.1 | 20.3 | 17.8 |
| ketoglutaricum | | | | | | | | |
| Rhodococcus | 47072 | 26 | — | 0 | 0 | 1 | 2 | 0 |
| erythropolis | | | | | | | | |
| Rhodococcus | 55899 | 30 | 7.8 | 2 | 7.4 | 12.5 | 11.1 | 13.9 |
| sp. DAP 96253 | | | | | | | | |
| Rhodococcus | 55898 | 26 | 8.2 | 7.8 | 10.1 | 12.3 | 10 | 13.8 |
| rhodochrous | | | | | | | | |
| DAP 96622 | | | | | | | | |

### Example 9

### Induction of Asparaginase I Activity in Rhodococcus sp. DAP 96253 Cells

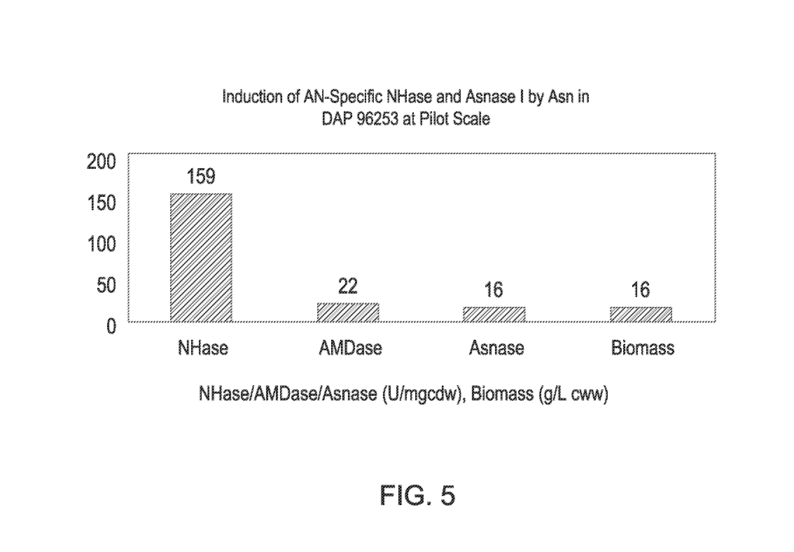

Rhodococcus sp. DAP 96253 were grown using biphasic medium as the source of inoculum for a 20 liter fermentation. The supplemental addition of medium/carbohydrate (either YEMEA, dextrose or maltose) was made to the modified R2A medium, containing cottonseed hydrolysate substituted for the Proteos Peptone 3 (PP3). Asparagine (0.15M solution) was added at a continuous rate of 1000 μl/min beginning at t=10 hour. At the end of the fermentation run, 159 units per milligram cell dry weight of acrylonitrile specific nitrile hydratase, 22 units of amidase per milligram cell dry weight, and 16 g/l cell packed wet weight were produced. The amount of various enzymes produced is provided in FIG. 5. As can be seen therein, 159 units of nitrile hydratase, 22 units of acrylamidase, and 16 units of asparaginase I per milligram cell dry weight was produced by the DAP 96253 cells.

### Example 10

### Effect of Media Composition on Asparaginase I Production in Rhodococcus sp. DAP 96253 Cells

Testing was performed to evaluate the effect on asparaginase I activity based upon the inducer used. In particular, testing was performed using asparagine, glutamine, succinonitrile, and isovaleronitrile as inducers (all added at 1000 ppm each). As can be seen in Table 8, asparagine was able to induce asparaginase I activity of 24.6 units/mg cell dry weight. Glutamine or succinonitrile also showed an ability to induce asparaginase I activity. Higher asparaginase I activity was obtained when maltose was added to YEMEA. The inclusion of Cobalt (5-50 ppm) in the medium also resulted in improvements when combined with either glucose or maltose.

TABLE 8
Asparaginase I levels in Rhodococcus sp. DAP 96253 Grown in
Medium with Carbohydrate Supplement

| | | | YEMEA - Maltose | |
|---|---|---|---|---|
| | YEMEA - Glucose | | Without | |
| Inducer | Without Cobalt | With Cobalt | Cobalt | With Cobalt |
| Asparagine | 5.3 | 6.5 | 8.7 | 24.6 |
| Glutamine | 1.5 | 1.9 | 9.3 | 8.1 |
| Succinonitrile | 6.5 | 8.5 | 11.0 | 10.0 |
| Isovaleronitrile | 3.5 | 2.9 | 6.8 | 7.0 |

### Example 11

### Effect of Trehalose on Nitrile Hydratase Stability

Testing was performed to evaluate nitrile hydratase stability in cells induced for nitrile hydratase activity using trehalose in the culture medium. The testing specifically compared the stabilization provided by the addition of trehalose to the culture medium. Rhodococcus sp., strain DAP 96253 was grown under various culture conditions and levels of trehalose (cellular and lipid bound) were measured. The levels of trehalose are provided below in Table 9. The greatest level of cellular trehalose is achieved when both trehalose and maltose are added to the culture medium.

TABLE 9
Cellular and lipid bound trehalose present in Rhodococcus sp., strain DAP
96253 cells grown on YEMEA supplemented with different sugars and
inducers.

| | Cellular | Lipid Bound | Total Trehalose |
|---|---|---|---|
| | Trehalose | Trehalose (mg/g | (cellular and lipid |
| Media | (mg/g cdw) | cdw) | bound) |
| G, Co, U | 2.50 | 0.980 | 3.48 |
| F, Co, U | 1.44 | 1.10 | 2.54 |
| M, Co, U | 2,90 | 0.99 | 3.89 |
| MD, Co, U | 3.00 | 1.35 | 4.35 |
| G, Co, U, ASN | 2.70 | 2.76 | 5.46 |
| F, Co, U, ASN | 3.17 | 4.70 | 7.87 |
| M, Co, U, ASN | 7.65 | 1.03 | 8.68 |
| MD, Co, U, ASN | 10.41 | 2.10 | 12.51 |
| G, Co, U, Tre | 4.8 | 2.08 | 6.88 |
| F, Co, U, Tre | 1.7 | 1.35 | 3.05 |
| M, Co, U, Tre | 42.20 | 5.00 | 47.20 |
| MD, Co, U, Tre | 42.00 | 5.22 | 47.22 |

G: YEMEA supplemented with glucose (4 g/L);
F: YEMEA supplemented with fructose (4 g/L);
M: YEMEA supplemented with maltose (4 g/L);
MD: YEMEA supplemented with maltodextrin (4 g/L);
Co: Cobalt (50 mg/L);
U: Urea (7.5 g/L);
ASN: Asparagine (1 g/L);
Tre: Trehalose (4 g/L).

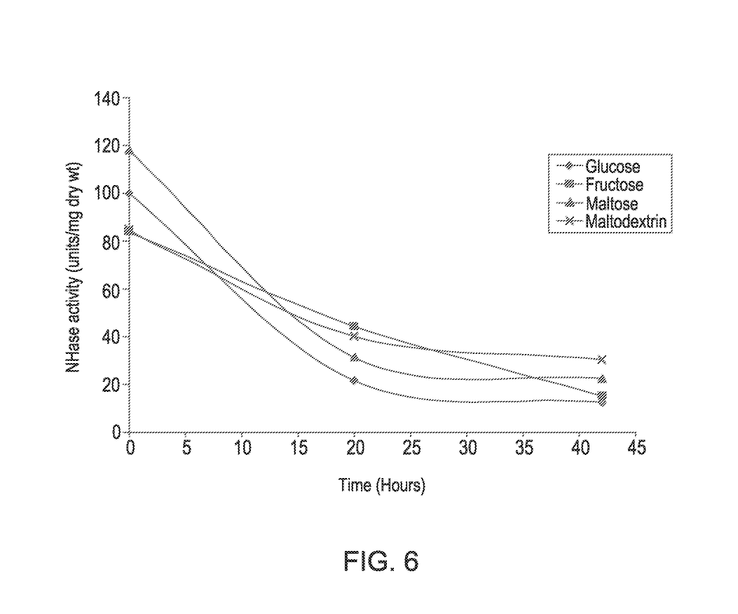

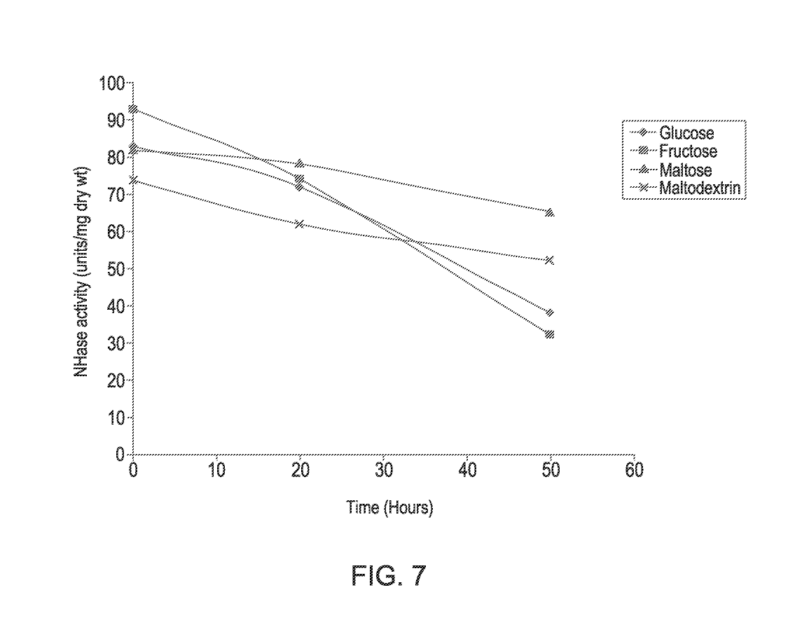

Further, as seen in FIGS. 6 and 7, nitrile hydratase activity is stabilized in Rhodococcus sp., strain DAP 96253 cells grown in the presence of trehalose. Under all growth conditions tested, the incorporation of trehalose significantly improved the thermal stability and, therefore, the effective half-life of nitrile hydratase present in Rhodococcus sp., strain DAP 96253 cells.

The medium used to obtain high levels of trehalose, in Rhodococcus sp., strain DAP 96253 cells contained 4 grams of trehalose per liter, whereas in stabilizing proteins or cells, concentrations in excess of 100 grams of trehalose per liter may be used.

It has previously been demonstrated that proteins supplemented with trehalose have been stabilized post recovery. Further, freeze-dried cells or dried cells have been improved post recovery through the addition of trehalose. As described herein, proteins were stabilized from the time of synthesis through protein recovery by increasing the cellular level of trehalose as well as the level of trehalose in the culture medium. This provided the benefits of trehalose protection and stabilization for the protein from the time of synthesis through the time of recovery. Further, addition of trehalose to the culture medium improved cellular stability, which is important when using the Rhodococcus cell as a matrix in which enzymes, such as nitrile hydratase are immobilized. Thus, both the protein and the protein producing cell, which becomes the catalyst platform, are simultaneously stabilized.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

1. A method for inducing an enzyme activity selected from the group consisting of nitrile hydratase activity, amidase activity, asparaginase I activity, and combinations thereof in a nitrile hydratase producing microorganism comprising culturing the nitrile hydratase producing microorganism in a medium comprising trehalose and one or more amide containing amino acids.

2. The method of claim 1, wherein the nitrile hydratase producing microorganism comprises bacteria selected from the group consisting of genus Rhodococcus, genus Brevibacterium, genus Pseudomonas, genus Pseudonocardia, genus Nocardia, and combinations thereof.

3. The method of claim 1, wherein the enzyme activity includes nitrile hydratase activity.

4. The method of claim 1, wherein the nitrile hydratase producing microorganism comprises bacteria from the genus Rhodococcus.

5. The method of claim 1, wherein the nitrile hydratase producing microorganism comprises bacteria selected from the group consisting of Rhodococcus rhodochrous DAP 96622, Rhodococcus sp. DAP 96253, and combinations thereof.

6. (canceled)

7. The method of claim 1, wherein the trehalose is present at a concentration of at least 1 gram per liter to 10 grams per liter of medium.

8. The method of claim 1, wherein the one or more amide containing amino acids are selected from the group consisting of asparagine, glutamine, asparagine derivatives, glutamine derivatives, and combinations thereof.

9. The method of claim 8, wherein the amide containing amino acids include asparagine and asparagine derivatives, and wherein the asparagine and asparagine derivatives include natural forms of asparagine, anhydrous asparagine, asparagine monohydrate, and L-isomers and D-isomers thereof.

10. The method of claim 8, wherein the amide containing amino acids include glutamine and glutamine derivatives, and wherein the glutamine and glutamine derivatives include natural forms of glutamine, anhydrous glutamine, glutamine monohydrate, and L-isomers and D-isomers thereof.

11. (canceled)

12. (canceled)

13. The method of claim 1, wherein the one or more amide containing amino acids are present at a concentration of 200 ppm to 2000 ppm.

14. The method of claim 1, wherein the one or more amide containing amino acids are present at a concentration of 50 parts per million (ppm) to 5000 ppm.

15. The method of claim 1, wherein the medium is free of any nitrile containing compounds.

16. The method of claim 1, wherein the induced nitrile hydratase producing microorganism has an enzyme activity greater than or equal to the activity of the same enzyme when induced in a medium comprising a nitrile containing compound.

17. The method of claim 1, wherein the induced nitrile hydratase producing microorganism has an enzyme activity that is at least 5% greater than the activity of the same enzyme when induced in a medium comprising a nitrile containing compound.

18. The method of claim 1, wherein the medium further comprises cobalt, urea, maltose, maltodextrin, or combinations thereof.

19. (canceled)

20. (canceled)

21. The method of claim 1, wherein the nitrile hydratase producing microorganisms are at least partially immobilized.

22. A method for stabilizing desired activity in an enzyme or a microorganism capable of producing the enzyme comprising contacting the enzyme or microorganism capable of producing the enzyme with a composition comprising trehalose and one or more amide containing amino acids, wherein the enzyme is selected from the group consisting of nitrile hydratase, amidase, and asparaginase I.

23. (canceled)

24. The method of claim 22, wherein the trehalose is present at a concentration of at least 1 grams per liter to 10 grams per liter.

25. (canceled)

26. The method of claim 22, wherein the one or more amide containing amino acids are present in a concentration of at least 50 ppm to 5000 ppm.

27. The method of claim 22, wherein the one or more amide containing amino acids are present at a concentration of 200 ppm to 2000 ppm.

28. The method of claim 22, wherein the amide containing amino acids are selected from the group consisting of asparagine, glutamine, asparagine derivatives, glutamine derivatives, and combinations thereof.

29. The method of claim 22, wherein the amide containing amino acids include asparagine and asparagine derivatives, and wherein the asparagine and asparagine derivatives include natural forms of asparagine, anhydrous asparagine, asparagine monohydrate, and L-isomers and D-isomers thereof.

30. The method of claim 22, wherein the amide containing amino acids include glutamine and glutamine derivatives, and wherein the glutamine and glutamine derivatives include natural forms of glutamine, anhydrous glutamine, glutamine monohydrate and L-isomers and D-isomers thereof.

31. The method of claim 22, wherein the desired activity of the enzyme or the microorganism capable of producing the enzyme is stabilized such that the desired activity after a time of at least 30 days at a temperature of 25° C. is maintained at a level of at least 50% of the initial activity exhibited by the enzyme or the microorganism capable of producing the enzyme.

32. The method of claim 22, wherein the microorganism comprises bacteria selected from the genus Rhodococcus, genus Brevibacterium, genus Pseudomonas, genus Pseudonocardia, genus Nocardia, and combinations thereof.

33. The method of claim 22, wherein the microorganism comprises bacteria selected from the group consisting of Rhodococcus rhodochrous DAP 96622, Rhodococcus sp. DAP 96253 and combinations thereof.

34. The method of claim 22, wherein the composition is free of any nitrile containing compounds.

35. The method of claim 22, wherein the composition further comprises cobalt, urea, maltose, maltodextrin, and combinations thereof.

36. The method of claim 22, wherein the microorganism is at least partially immobilized.

37. A composition comprising:
(a) a nutrient medium comprising trehalose and one or more amide containing amino acids;
(b) one or more microorganisms capable of producing one or more enzymes selected from the group consisting of nitrile hydratase, amidase, asparaginase I, and combinations thereof; and
(c) one or more enzymes selected from the group consisting of nitrile hydratase, amidase, asparaginase I, and combinations thereof.

38. The composition of claim 37, wherein the one or more microorganisms comprise bacteria selected from the group consisting of genus Rhodococcus, genus Brevibacterium, genus Pseudomonas, genus Pseudonocardia, genus Nocardia, and combinations thereof.

39. (canceled)

40. The composition of claim 37, wherein the trehalose is present at a concentration of at least 1 grams per liter to 10 grams per liter of medium.

41. The composition of claim 37, wherein the one or more amide containing amino acids are selected from the group consisting of asparagine, glutamine, asparagine derivatives, glutamine derivatives, and combinations thereof.

42. The composition of claim 37, wherein the amide containing amino acids include asparagine and asparagine derivatives, and wherein the asparagine and asparagine derivatives include natural forms of asparagine, anhydrous asparagine, asparagine monohydrate, and L-isomers and D-isomers thereof.

43. The composition of claim 37, wherein the amide containing amino acids include glutamine and glutamine derivatives, and wherein the glutamine and glutamine derivatives include natural forms of glutamine, anhydrous glutamine, glutamine monohydrate, and L-isomers and D-isomers thereof.

44. (canceled)

45. (canceled)

46. The composition of claim 37, wherein the one or more amide containing amino acids are present in a concentration of 200 ppm to 2000 ppm.

47. The composition of claim 37, wherein the one or more microorganisms comprise bacteria selected from the genus Rhodococcus.

48. The composition of claim 37, wherein the one or more microorganisms comprise bacteria selected from the group consisting of Rhodococcus rhodochrous, Rhodococcus sp. DAP 96253, Brevibacterium ketoglutaricum, and combinations thereof.

49. The composition of claim 37, wherein the one or more microorganisms are at least partially immobilized.

50. The composition of claim 37, wherein the medium further comprises cobalt, urea, maltose, maltodextrin, or combinations thereof.

51. (canceled)

52. The composition of claim 37, wherein the medium is free of any nitrile containing compounds.

53. (canceled)

54. A method for delaying a plant development process comprising exposing a plant or plant part to one or more enzymes, wherein the enzymes are produced by one or more bacteria by culturing the bacteria in a medium comprising trehalose and one or more amide containing amino acids, and wherein the enzymes are exposed to the plant or plant part in a quantity sufficient to delay the plant development process.

55. A method for delaying a plant development process comprising exposing a plant or plant part to an enzymatic extract of one or more bacteria, wherein the bacteria are cultured in a medium comprising trehalose and one or more amide containing amino acids, and wherein the enzymatic extract is exposed to the plant or plant part in a quantity sufficient to delay the plant development process.


SEQUENCE LISTING (13 sequences)

SEQ ID NO: 1 - protein, 229 aa, Rhodococcus sp.
MDGIHDTGGMTGYGPVPYQKDEPFFHYEWEGRTLSILTWMHLKGMSWWDKSRFFRESMGNENYVNEIRNSYYTHWLSAAERILVADKIITEEERKHRVQEILEGRYTDRNPSRKFDPAEIEKAIERLHEPHSLALPGAEPSFSLGDKVKVKNMNPLGHTRCPKYVRNKIGEIVTSHGCQIYPESSSAGLGDDPRPLYTVAFSAQELWGDDGNGKDVVCVDLWEPYLISA

SEQ ID NO: 2 - protein, 229 aa, Nocardia sp.; identical to SEQ ID NO: 1

SEQ ID NO: 3 - protein, 229 aa, Rhodococcus rhodochrous
  Uncultured bacterium BD2
MDGIHDTGGMTGYGPVPYQKDEPFFHYEWEGRTLSILTWMHLKGISWWDKSRFFRESMGNENYVNEIRNSYYTHWLSAAERILVADKIITEEERKHRVQEILEGRYTDRKPSRKFDPAQIEKAIERLHEPHSLALPGAEPSFSLGDKIKVKSMNPLGHTRCPKYVRNKIGEIVAYHGCQIYPESSSAGLGDDPRPLYTVAFSAQELWGDDGNGKDVVCVDLWEPYLISA

SEQ ID NO: 4 - protein, 166 aa, Unknown
  Uncultured bacterium BD2
MDGIHDTGGMTGYGPVPYQKDEPFFHYEWEGRTLSILTWMHLKGISWWDKSRFFRESMGNENYVDEIRNSYYTHWLSAAERILVADKIITEEERKHRVQEILEGRYTDRKPSRKFDPAQIEKAIERLHEPHSLALPGAEPSFSLGDKNQSEEYEPAGTHTVPEICA

SEQ ID NO: 5 - protein, 203 aa, Rhodococcus sp.
MSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEDELAKLVSRDSMIGVSNALTPQEVIV

SEQ ID NO: 6 - protein, 203 aa, Rhodococcus rhodochrous
MSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEEELTKLVSRDSMIGVSNALTPQEVIV

SEQ ID NO: 7 - protein, 180 aa, Unknown
  Uncultured bacterium SP1 <220<
MSEHVNKYTEYEARTKAVETLLYERGLITPAAVDRVVSYYENEIGPMGGAKVVAKSWVDPEYRKWLEEDATAAMASLGYAGEQAHHVVVCTLCSCYPWPVLGLPPAWYKSMEYRSRVVADPRGVLKRDFGFDIPDEVEVRVWDSSSEIRYIVIPERPAGTDGWSEEELTKLVSRDSIIGV

SEQ ID NO: 8 - protein, 345 aa, Rhodococcus rhodochrous
MRHGDISSSPDTVGVAVVNYKMPRLHTKADVLENARAIAKMVVGMKAGLPGMDLVVFPEYSTMGIMYDNDEMYATAATIPGDETDIFAQACRDAKTWGVFSITGERHEDHPNKPPYNTLVLINDQGEIVQKYRKILPWTPIEGWYPGGQTYVTDGPKGLKISLIICDDGNYPEIWRDCAMKGAELIVRPQGYMYPSKEQQVLMAKAMAWANNCYVAVANATGFDGVYSYFGHSAIIGFDGRTLGECGEEDYGVQYAQLSLSTIRDARANDQSQNHLFKLLHRGYTGVFAGGDGDKGVADCPFDFYRNWVNDAEATQKAVEAITRETIGVADCPVYDLPSEKTMDA

SEQ ID NO: 9 - protein, 345 aa, Nocardia farcinica
MRHGDISSSPDTVGVAVVNYKMPRLHTKAEVLDNCRRIADMLVGMKSGLPGMDLVVFPEYSTQGIMYDEQEMYDTAATVPGEETAIFSAACREAGVWGVFSITGEQHEDHPRKPPYNTLVLIDDHGEIVQKYRKILPWCPIEGWYPGDTTYVTEGPKGLKISLIVCDDGNYPEIWRDCAMKGAELIVRCQGYMYPSKDQQVLMAKAMAWANNCYVAVANAAGFDGVYSYFGHSALIGFDGRTLGETGEEEYGIQYAQLSISAIRDARAHDQSQNHLFKLLHRGYSGVHAAGDGDRGVADCPFEFYKLWVTDAQQARERVEAITRDTVGVADCRVGSLPVEQTLEA

SEQ ID NO: 10 - protein, 346 aa, Pseudomonas aeruginosa [sequence omitted]

SEQ ID NO: 11 - protein, 339 aa, Helicobacter pylori
MRHGDISSSPDTVGVAVVNYKMPRLHTKNEVLENCRNIAKVIGGVKQGLPGLDLIIFPEYSTHGIMYDRQEMFDTAASVPGEETAILAEACKKNKVWGVFSLTGEKHEQAKKNPYNTLILVNDKGEIVQKYRKILPWCPIECWYPGDKTYVVDGPKGLKVSLIICDDGNYPEIWRDCAMRGAELIVRCQGYMYPAKEQQIAIVKAMAWANQCYVAVANATGFDGVYSYFGHSSIIGFDGHTLGECGEEENGLQYAQLSVQQIRDARKYDQSQNQLFKLLHRGYSGVFASGDGDKGVAECPFEFYKTWVNDPKKAQENVEKFTRPSVGVAACPVGDLPTK

SEQ ID NO: 12 - protein, 339 aa, Helicobacter pylori
MRHGDISSSPDTVGVAVVNYKMPRLHTKNEVLENCRNIAKVIGGVKQGLPGLDLIIFPEYSTHGIMYDRQEMFDTAASVPGEETAIFAEACKKNKVWGVFSLTGEKHEQAKKNPYNTLILVNDKGEIVQKYRKILPWCPIECWYPGDKTYVVDGPKGLKVSLIICDDGNYPEIWRDCAMRGAELIVRCQGYMYPAKEQQIAIVKAMAWANQCYVAVANATGFDGVYSYFGHSSIIGFDGHTLGECGEEENGLQYAQLSVQQIRDARKYDQSQNQLFKLLHRGYSGVFASGDGDKGVAECPFEFYKTWVNDPKKAQENVEKITRPSVGVAACPVGDLPTK

SEQ ID NO: 13 - protein, 346 aa, Pseudomonas aeruginosa
MRHGDISSSNDTVGVAVVNYKMPRLHTAAEVLDNARKIADMIVGMKQGLPGMDLVVFPEYSLQGIMYDPAEMMETAVAIPGEETEIFSRACRKANVWGVFSLTGERHEEHPRKAPYNTLVLIDNNGEIVQKYRKIIPWCPIEGWYPGGQTYVSEGPKGMKISLIICDDPNYPEIWRDCAMKGAELIVRCQGYMYPAKDQQVMMAKAMAWANNCYVAVANAAGFDGVYSYFGHSAIIGFDGRTLGECGEEEMGIQYAQLSLSQIRDARANDQSQNHLFKILHRGYSGLQASGDGDRGLAECPFEFYRTWVTDAEKARDNVERLTRSTTGVAQCPVGRLPYEGLEKEA